(19) United States
(12) Patent Application Publication
(10) Pub. No.: US 2007/0269510 A1
(43) Pub. Date: Nov. 22, 2007

(54) Solid Unit Dosage Forms of 5-Ht1 Agonist

(76) Inventors: Sudarshan Nimbalkar, Hyderabad (IN); Kishor Dattatray Deo, Hyderabad (IN); Hidaytulla Shamshuddin Aga, Hyderabad (IN); Sivakumaran Meenakshisunderam, Hyderabad (IN)

Correspondence Address: Jay R Akhave, 2058 N Mills Ave # 612, Claremont, CA, 91711, US

(21) Appl. No.: 11/662,619

(22) PCT Filed: Sep. 26, 2005
(86) PCT No.: PCT/IB05/03100

§ 371 Date: Mar. 12, 2007

(30) Foreign Application Priority Data
Sep. 29, 2004 (IN) 998/CHE/2004

(51) Int. Cl. A61K 9/20; A61K 31/403; A61P 25/06

(52) U.S. Cl. 424/465; 514/415

(57) ABSTRACT

The present invention relates to pharmaceutical compositions of 5-HT₁ agonist. More particularly, the present invention relates to uncoated tablets of sumatriptan succinate. The present invention also relates to a process for the preparation of uncoated tablets of sumatriptan succinate.

### FIELD OF THE INVENTION

The present invention relates to pharmaceutical compositions of 5-HT₁ agonist. More particularly, the present invention relates to uncoated tablets of sumatriptan succinate.

The present invention also relates to a process for the preparation of uncoated tablets of sumatriptan succinate.

### BACKGROUND OF THE INVENTION

Sumatriptan and its acid salts, particularly the succinate salt, are selective 5-hydroxytryptamine-1 agonists. It is indicated for the acute treatment of migraine attacks with or without aura in adults. Chemically, Sumatriptan is 3-[2-(dimethylamino)ethyl]-N-methyl-indole-5-methanesulfonamide and is marketed as its succinate salt under the trade name IMITREX® in US and IMIGRAN® IN Europe. Sumatriptan and its succinate salt is disclosed specifically in U.S. Pat. No. 5,037,845.

Sumatriptan and its pharmaceutically acceptable salts have an unpleasant taste profile and, when administered orally, may intensify the nausea and vomiting associated with migraines. This limits the use of sumatriptan orally, which is considered to be the most widely accepted and convenient route of administration.

U.S. Pat. No. 5,863,559 discloses film-coated tablets of sumatriptan. The core is substantially covered with a coating that includes film-forming polymers, such as hydroxypropylmethylcellulose, hydroxypropylcellulose or methylcellulose, and copolymers of methacrylic acid and methyl methacrylate polymers.

WO 01/37816 discloses a process for the coating of sumatriptan tablet cores and tablets to provide taste masking of the sumatriptan. The process includes spraying a coating solution or suspension of a sugar, a starch, or a mixture of a sugar and a starch, onto tablet cores to obtain coated tablets. There is the proviso that film-forming agents in the suspension or solution are excluded.

WO 2004/009085 discloses uncoated, taste-masked sumatriptan tablet comprising: an intragranular portion comprising granules of sumatriptan or a pharmaceutically acceptable salt and one or more diluents and/or binders present in a sufficient amount to cause taste-masking of the sumatriptan or pharmaceutically acceptable salt; and an extragranular portion comprising one or more pharmaceutically acceptable excipients around the intragranular granules. This publication further discloses wax polishing of sumatriptan tablet. The wax polishing includes spraying a solution or suspension of wax material onto the tablet and/or sprinkling a powder grade wax onto the tablet.

The wax coating is primarily a hydrophobic layer, which can inhibit the penetration of the fluids into the tablet cores and release the content in to the medium for absorption. Further, it is a tedious process to control the amount of solution or suspension of sprayed onto the tablets, which may result in non-uniform release of the drug. Non-uniform release of the active ingredient may create bioavailability related problems. Hence, there exists a need to develop uncoated tablets, which have better advantages over the coated tablets.

WO 2005/70417 discloses a taste masking pharmaceutical composition for oral administration, comprising a core of active ingredient and one or more outer non-active taste masking layers formed on the core by application of pressure.

Film-forming agents are usually polymers, which form a continuous, elastic and uniform covering around the tablet core, which is at least partially detachable as a continuous layer. Such a film in a film-coated tablet, however, may provide a considerable barrier to the penetration of aqueous fluids into the tablet cores, which is a pre-requisite for disintegration of the tablet core and release of the pharmaceutically active compound. Insufficient release of the pharmaceutically active compound may create bioavailability problems. Examination and control of such thin layers is difficult affording special and complex, but non-specific, testing methods. Varying film thickness in different film-coated tablets within the same batch may not be excluded.

In the prior art, a coating over the core tablet has been used to mask the bitter taste of sumatriptan. Though the coating may mask the unpleasant taste, if the thickness and composition of the coating is not properly controlled it may affect the disintegration and dissolution characteristics of the tablet. Further, the coating operation is a highly controlled and costly process. Hence, there exists need to develop uncoated tablets, which have better advantages over the coated tablets.

### OBJECTIVE OF THE INVENTION

Accordingly, the main objective of present invention is to provide uncoated tablet of sumatriptan in such a way that it will comply with the reference product in terms of in vivo parameters like bioequivalence and in vitro parameters like dissolution, disintegration and etc.

Yet another objective of the present invention is to provide taste masked uncoated tablets of sumatriptan, thereby avoiding the costly coating process.

Yet another objective of the present invention is to provide a process to prepare uncoated tablets of sumatriptan on a commercial scale with adequate hardness and good reproducibility.

### SUMMARY OF THE INVENTION

According to the main embodiment of the present invention, there is provided uncoated tablets of sumatriptan or its pharmaceutically acceptable salts, comprising intragranular portion containing sumatriptan and its pharmaceutically acceptable salts and optionally disintegrant and/or surfactant and an extragranular portion comprising diluent, an alkaline agent, disintegrant and lubricant.

In yet another embodiment of the present invention, there is provided a process for the preparation of uncoated tablets of sumatriptan comprising the steps of granulating sumatriptan or a pharmaceutically acceptable salt with or without disintegrant and surfactant; mixing the granules with diluent, an alkaline agent, disintegrant, lubricant and finally compressing the mixed blend into a tablet.

In yet another embodiment of the present invention, there is provided uncoated tablets of sumatriptan or its pharmaceutically acceptable salt free of binder and/or diluents in the intragranular portion.

### DETAILED DESCRIPTION OF THE INVENTION

The term pharmaceutically acceptable salts as used herein includes inorganic or organic acids such as hydrochloride, hydrobromide, sulphate, nitrate, phosphate, formate, mesylate, citrate, benzoate, fumarate, maleate, tartrate and succinate.

In yet another embodiment, the uncoated tablets of sumatriptan further comprise one or more sweetening agents or colorants.

The diluents used according to the present invention are selected from calcium phosphate-dibasic, calcium carbonate, cellulose-microcrystalline, cellulose powdered, calcium silicate, kaolin, starch, lactose, sucrose, starch pregelatinized, polyols such as mannitol, sorbitol, lactitol, xylitol, maltitol, sucrose and combinations thereof.

Suitable disintegrants used in accordance with the present invention are selected from croscarmellose sodium, crospovidone, sodium starch glycolate, sodium carboxymethylcellulose, calcium carboxymethylcellulose, hydroxypropylcellulose, xanthan gum, alginic acid, alginates, carbopols and the like or combination thereof, preferably croscarmellose sodium, crospovidone, sodium starch glycolate.

Suitable surfactants used in accordance with the present invention are selected from polyoxyethylene sorbitan fatty acid esters (polysorbates), sodium lauryl sulfate, docusate sodium and the like or combination thereof.

Suitable lubricants according to the present invention are selected from talc, magnesium stearate, stearic acid, sodium stearyl fumarate, hydrogenated vegetable oil or glyceryl behenate, and suitable glidants include colloidal silicon dioxide or talc, preferably colloidal silicon dioxide.

The alkaline agent used in the present invention is selected from carbonate or bicarbonates of potassium, sodium or calcium.

Suitable sweeteners used are selected from glucose, glycerol, fructose, sucrose, lactose, maltose, sorbitol, xylitol, maltitol, erythritol, aspartame, prosweet and the like.

In yet another embodiment of the present invention, there is provided a process for the preparation of uncoated tablets of sumatriptan or its pharmaceutically acceptable salts, comprising intragranular portion containing sumatriptan and its pharmaceutically acceptable salts and optionally disintegrant and/or surfactant and an extragranular portion comprising diluent, an alkaline agent, disintegrant and lubricant, comprising the steps of:

i) dry blending sumatriptan with or without disintegrants and/or surfactant,

ii) granulating the blend obtained in step (i) with a solvent,

iii) mixing the granules of step (ii) with diluent, an alkaline agent, disintegrant, lubricant and

iv) compressing the mixed blend into tablet.

The solvents used for preparing granules can be an aqueous and/or non-aqueous solvent. The non-aqueous solvent selected from alcohol or isopropyl alcohol.

The process described herein avoids coating step and thereby reduce the processing time and costs associated with coating. Moreover, absence of any coating over the tablets helps to achieve the desired disintegration and dissolution characteristics without failure.

In yet another embodiment, there is provided a method of treating a human suffering from a migraine condition. The method includes orally administering an uncoated, tablet of sumatriptan that includes an intragranular portion and an extragranular portion. The intragranular portion includes granules of sumatriptan or a pharmaceutically acceptable salt and optionally disintegrant and surfactant. The extragranular portion includes diluent, an alkaline agent, disintegrant, lubricant around the intragranular granules.

In yet another embodiment, the tablet contains about 10 mg to 200 mg of sumatriptan.

The following examples further exemplify the inventions and are not intended to limit the scope of the inventions. It is obvious to those skilled in the art to find out the composition for other dosage forms and substitute the equivalent excipients as described in this specification or with the one known to the industry.

### EXAMPLE 1

Formulation of uncoated tablets of sumatriptan without using
disintegrant or surfactant

| Ingredients | Quantity mg/tablet |
|---|---|
| Intra granular | |
| Sumatriptan succinate eq. to | 140.00 |
| Sumatriptan 100 mg | |
| Purified water | Qs |
| Extra granular | |
| Dicalcium phosphate | 117.00 |
| Microcrystalline cellulose | 59.00 |
| Sodium bicarbonate | 15.00 |
| Croscarmellose sodium | 4.50 |
| Magnesium stearate | 4.50 |
| Average weight | 340.00 |

The processing steps that are involved in making uncoated tablets of sumatriptan disclosed above are given below:—

i) sumatriptan succinate was granulated using purified water to get a cohesive mass of desired consistency,

ii) dried and sieved to obtain uniform particle size through a suitable mesh,

iii) dried and sieved granules of step (ii) were mixed with extra granular dicalcium phosphate, microcrystalline cellulose, croscarmellose sodium, sodium bicarbonate and magnesium stearate through a suitable mesh and

iv) compressed the blend of step (iii) into tablets.

The processing steps that are involved in making uncoated tablets of sumatriptan disclosed in examples 2-7 are as given below:—

i) sumatriptan succinate and full or part of disintegrant and/or surfactant were sifted and mixed.

ii) the blend from step 1 was granulated using purified water and dried the granules,

iii) dried granules of step (ii) were sieved through a suitable mesh to get uniform granules,

iv) granules of step (iii) were mixed with extragranular ingredients and

v) compressed the blend from step (iv) into tablets.

### EXAMPLE 2

Formulation of uncoated tablets of sumatriptan with disintegrant

| Ingredients | Quantity mg/tablet |
|---|---|
| Intra granular | |
| Sumatriptan succinate eq. to | 140.0 |
| Sumatriptan 100 mg | |
| Croscarmellose sodium | 20.00 |
| Purified water | Qs |
| Extra granular | |
| Dicalcium phosphate | 112.00 |
| Microcrystalline cellulose | 46.50 |
| Sodium bicarbonate | 15.00 |
| Croscarmellose sodium | 2.00 |
| Magnesium stearate | 4.50 |
| Average weight | 340.0 |

### EXAMPLE 3

| Ingredients | Quantity mg/tablet |
|---|---|
| Intra granular | |
| Sumatriptan Succinate eq. to | 140.0 |
| Sumatriptan 100 mg | |
| Sodium starch Glycolate | 20.00 |
| Purified water | Qs |
| Extra granular | |
| Dicalcium phosphate | 112.00 |
| Microcrystalline cellulose | 46.50 |
| Sodium bicarbonate | 15.00 |
| Croscarmellose sodium | 2.00 |
| Magnesium stearate | 4.50 |
| Average weight | 340.0 |

### EXAMPLE 4

Formulation of uncoated tablets of Sumatriptan with disintegrating agent
and surfactant

| Ingredients | Quantity mg/tablet |
|---|---|
| Intra granular | |
| Sumatriptan succinate eq. to | 140.00 |
| Sumatriptan 100 mg | |
| Croscarmellose sodium | 20.00 |
| Polysorbate 80 | 1.40 |
| Purified water | Qs |
| Extra granular | |
| Dicalcium phosphate | 107.10 |
| Microcrystalline cellulose | 45.00 |
| Sodium bicarbonate | 20.00 |
| Croscarmellose sodium | 2.00 |
| Magnesium stearate | 4.50 |
| Average weight | 340.00 |

### EXAMPLE 5

| Ingredients | Quantity mg/tablet |
|---|---|
| Intra granular | |
| Sumatriptan Succinate eq. to | 140.00 |
| Sumatriptan 100 mg | |
| Croscarmellose sodium | 20.00 |
| Sodium lauryl Sulphate | 1.40 |
| Purified water | Qs |
| Extra granular | |
| Dicalcium phosphate | 107.10 |
| Microcrystalline cellulose | 45.00 |
| Sodium bicarbonate | 20.00 |
| Croscarmellose sodium | 2.00 |
| Magnesium stearate | 4.50 |
| Average weight | 340.00 |

### EXAMPLE 6

Formulation of uncoated tablets of sumatriptan with surfactant

| Ingredients | Quantity mg/tablet |
|---|---|
| Intra granular | |
| Sumatriptan succinate eq. to | 140.00 |
| Sumatriptan 100 mg | |
| Polysorbate 80 | 4.20 |
| Purified water | Qs |
| Extra granular | |
| Dicalcium phosphate | 124.30 |
| Microcrystalline cellulose | 45.00 |
| Sodium bicarbonate | 20.00 |
| Croscarmellose sodium | 2.00 |
| Magnesium stearate | 4.50 |
| Average weight | 340.00 |

### EXAMPLE 7

| Ingredients | Quantity mg/tablet |
|---|---|
| Intra granular | |
| Sumatriptan Succinate eq. to | 140.00 |
| Sumatriptan 100 mg | |
| Docusate Sodium | 4.20 |
| Purified water | Qs |
| Extra granular | |
| Dicalcium phosphate | 124.30 |
| Microcrystalline cellulose | 45.00 |
| Sodium bicarbonate | 20.00 |
| Croscarmellose sodium | 2.00 |
| Magnesium stearate | 4.50 |
| Average weight | 340.00 |

Dissolution Profile:

The dissolution studies were performed in 900 ml of 0.01 M Hydrochloric acid, at 30 RPM by USP II method. The release profile (% of drug released in minutes) is given in table 1.

TABLE I

| | Time | % Drug release | | | | | | |
|---|---|---|---|---|---|---|---|---|
| S. No. | in min. | Ex 1 | Ex 2 | Ex 3 | Ex 4 | Ex 5 | Ex 6 | Ex 7 |
| 1 | 5 | 87 | 93 | 89 | 90 | 85 | 82 | 88 |
| 2 | 10 | 90 | 94 | 92 | 99 | 90 | 88 | 90 |
| 3 | 15 | 93 | 94 | 94 | 99 | 92 | 90 | 91 |
| 4 | 30 | 94 | 94 | 94 | 99 | 93 | 91 | 91 |
| 5 | 45 | 95 | 95 | 95 | 99 | 96 | 93 | 94 |

The stability study of Sumatriptan Succinate tablets at accelerated stability condition 40° C./75% RH was studied for a period of three months with the formulation and the data shows that the tablets are stable and there is no sign of degradation.

1. Uncoated tablets of sumatriptan or its pharmaceutically acceptable salt, comprising intragranular portion containing sumatriptan and its pharmaceutically acceptable salts and optionally disintegrant and/or surfactant and an extragranular portion comprising diluent, an alkaline agent, disintegrant and lubricant.

2. The tablet as claimed in claim 1, wherein the pharmaceutically acceptable salts is selected from hydrochloride, hydrobromide, sulphate, nitrate, phosphate, formate, mesylate, citrate, benzoate, fumarate, maleate, tartrate and succinate.

3. The tablet as claimed in claim 1, wherein the diluent is selected from calcium phosphate-dibasic, calcium carbonate, cellulose-microcrystalline, cellulose powdered, calcium silicate, kaolin, starch, lactose, sucrose, starch pregelatinized, polyols such as mannitol, sorbitol, lactitol, xylitol, maltitol, sucrose and combinations thereof.

4. The tablet as claimed in claim 1, wherein the disintegrants are selected from croscarmellose sodium, crospovidone, sodium starch glycolate, sodium carboxymethylcellulose, hydroxypropylcellulose, xanthan gum, alginic acid, alginates, carbopols or combination thereof, preferably croscarmellose sodium, crospovidone or sodium starch glycolate.

5. The tablets as claimed in claim 1, wherein the surfactant is selected from polyoxyethylene sorbitan fatty acid esters, sodium lauryl sulfate, docusate sodium or combination thereof.

6. The tablets as claimed in claim 1, wherein lubricant is selected from magnesium stearate, stearic acid, sodium stearyl fumarate, hydrogenated vegetable oil or glyceryl behenate.

7. The tablets as claimed in claim 1, wherein alkaline agent is selected from carbonate or bicarbonates of potassium, sodium or calcium.

8. A process for the preparation of uncoated tablets of sumatriptan as claimed in claim 1, wherein the processing steps include:
i) dry blending sumatriptan with or without disintegrant and/or
ii) granulating the blend obtained in step (i) with a solvent,
iii) mixing the granules of step (ii) with diluent, an alkaline agent, disintegrant, lubricant and
iv) compressing the mixed blend into tablet.

9. A method of treating migraine, which comprises administering the composition of claim 1.
